(19) United States
(12) Patent Application Publication
(10) Pub. No.: US 2006/0025671 A1
(43) Pub. Date: Feb. 2, 2006

(54) Image display apparatus, image display method and the program

(75) Inventor: Tetsuro Kusunoki, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/189,721

(22) Filed: Jul. 27, 2005

(30) Foreign Application Priority Data
Jul. 27, 2004 (JP) 218637/2004

(51) Int. Cl. A61B 5/05

(52) U.S. Cl. 600/407

(57) ABSTRACT

An image display apparatus and method capable of accurately retrieving images of comparable cases to that of an image to be diagnosed from multitudes of images. Data of a diagnostic image which has anatomical structures obtained by imaging a predetermined region of a subject to which a diagnosis is to be given. Then, a candidate image that has a candidate region of abnormal shadow is obtained, and the anatomical information and characteristic amounts, which indicate a likelihood of the candidate region being an abnormal shadow, are obtained from the candidate image. Thereafter, abnormal shadow images that have comparable anatomical information and characteristic amounts to those of the candidate region are retrieved from an image database and displayed comparably with the candidate region.

### BACKGROUND OF THE INVENTION

1. Field of the Invention

The present invention relates to an image display apparatus and method for displaying a comparable image to an object image. The present invention also relates to a program for causing a computer to perform the functions of the image display apparatus.

2. Description of the Related Art

In medical facilities including medical centers and general practitioner's offices, various types of medical images, such as x-ray, CT, and MR images are obtained and used for diagnoses of medical conditions, follow-up observations, assessments of curing or progress state of diseases. Normally, observers, such as doctors or the like, give diagnoses by observing the images and decide treatment courses of the patients or the like.

In medical facilities, most of these medical images used for diagnoses have been stored in the form of hard copies. In order to facilitate management of increasing number of medical images on a daily basis and reducing required space for storage, an image filing system is proposed. The system generates digital images from the medical images or the like, and keeps them on recording media, such as optical disks, magnetic disks, or the like.

In addition to reduced storage space for the images, the filing system affords the users a useful tool for managing the images in the form of image data files so that the images may be used in various ways. But, it is very difficult to find out adequate comparable cases to the diagnostic object from multitudes of past images stored in the form of image data files. Thus, there is a need for a method capable of finding out adequate comparable cases to the image of diagnostic object from multitudes of cases.

Under these circumstances, one such system is proposed as described, for example, in U.S. Patent Application Publication No. 20040003001. In the system, an image portion of a subject within the region of interest (ROI) set on the image is inputted, and a comparable image data file that includes a portion having comparable characteristics to those of the ROI image, and diagnostic data related to the comparable image data file are retrieved from a case database and read into the system. Thereafter, a similarity determining item, such as the “shape of shadow” or the like is selected to perform a similarity determining process for the selected item and the result is displayed on the monitor.

Another system is also proposed as described, for example, in U.S. Patent Application Publication No.20020065460. In the system, comparable reference images are retrieved from a database by checking characteristic amounts obtainable from reference images stored in the database and characteristic amounts obtainable from the location information of a lesion on a diagnostic image. Then, a probability of disease under each disease name of the reference images are calculated by referring to the remarks related to the reference images for which the similarity levels have been calculated. Thereafter, the reference images, disease names, and the probabilities are displayed in the order of the calculated probabilities so that a doctor giving a diagnosis by observing the diagnostic image may readily refer to the reference images having similarities to the diagnostic image, and the diagnostic accuracy may be improved.

In the latter system, however, the characteristics of a lesion, such as a lung cancer, appearing on the image vary according to the locations of the lesion within the lung field, or if it is overlapped with a rib or not. Thus, these images are inadequate as the reference images for giving diagnoses.

The former system retrieves comparable cases using a selected similarity determining item for ROI indicated by the observer, such as a doctor. But, anatomical structures of the background images are not taken into account. Thus, these images are inadequate as the reference images for giving diagnoses. Further, comparable images may be displayed when ROI is indicated by the observer, but if a lesion candidate is overlooked and ROI is not specified, there is no way to alert the observer.

### SUMMARY OF THE INVENTION

The present invention has been developed in view of the circumstances described above, and it is an object of the present invention to provide a comparable image display apparatus capable of accurately retrieving images of similar cases to that of a diagnostic image from multitudes of images.

The comparable image display apparatus of the present invention comprises:

an image database for storing medical images, each having an anatomical structure with an abnormal shadow, obtained by imaging a predetermined region of a subject, such as a human body or the like;

an obtaining means for obtaining data of a diagnostic image having anatomical structures obtained by imaging a subject, such as a human body or the like, to which a diagnosis is to be given;

a candidate image obtaining means for obtaining a candidate image from the data of the diagnostic image, the candidate image having a candidate region of abnormal shadow included in the diagnostic image;

an anatomical information obtaining means for obtaining anatomical information of the candidate region by detecting an anatomical structure included in the candidate image;

a characteristic amount obtaining means for obtaining characteristic amounts from the candidate region of abnormal shadow included in the candidate image, the characteristic amounts indicating a likelihood of the candidate region being an abnormal shadow;

a retrieving means for retrieving an abnormal shadow image from the image database, the abnormal shadow image being a medical image with an abnormal shadow having comparable anatomical information and characteristic amounts to the anatomical information and characteristic amounts of the candidate region obtained from the candidate image; and

a display means for comparably displaying the candidate region and the abnormal shadow image retrieved from the image database.

The comparable image display method according to an embodiment of the present invention comprises:

an obtaining step for obtaining data of a diagnostic image having anatomical structures obtained by imaging a subject, such as a human body or the like, to which a diagnosis is to be given;

a candidate image obtaining step for obtaining a candidate image from the data of the diagnostic image, the candidate image having a candidate region of abnormal shadow included in the diagnostic image;

an anatomical information obtaining step for obtaining anatomical information of the candidate region by detecting an anatomical structure included in the candidate image;

a characteristic amount obtaining step for obtaining characteristic amounts from the candidate region of abnormal shadow included in the candidate image, the characteristic amounts indicating a likelihood of the candidate region being an abnormal shadow;

a retrieving step for retrieving an abnormal shadow image from an image database storing medical images, each having an anatomical structure with an abnormal shadow, obtained by imaging a predetermined region of a subject, such as a human body or the like, the abnormal shadow image being a medical image with an abnormal shadow having comparable anatomical information and characteristic amounts to the anatomical information and characteristic amounts of the candidate region obtained from the candidate image; and

a displaying step for comparably displaying the candidate region and the abnormal shadow image retrieved from the image database.

The comparable image display method according to another embodiment of the present invention comprises:

an obtaining step for obtaining data of a diagnostic image having anatomical structures obtained by imaging a subject, such as a human body or the like, to which a diagnosis is to be given;

a candidate image obtaining step for obtaining a candidate image from the data of the diagnostic image, the candidate image having a candidate region of abnormal shadow included in the diagnostic image;

a characteristic amount obtaining step for obtaining characteristic amounts from the candidate region of abnormal shadow included in the candidate image, the characteristic amounts indicating a likelihood of the candidate region being an abnormal shadow;

an anatomical information obtaining step for obtaining anatomical information of the candidate region by detecting an anatomical structure included in the candidate image;

a retrieving step for retrieving an abnormal shadow image from an image database storing medical images, each having an anatomical structure with an abnormal shadow, obtained by imaging a predetermined region of a subject, such as a human body or the like, the abnormal shadow image being a medical image with an abnormal shadow having comparable anatomical information and characteristic amounts to the anatomical information and characteristic amounts of the candidate region obtained from the candidate image; and

a displaying step for comparably displaying the candidate region and the abnormal shadow image retrieved from the image database.

The program of the present invention is a program for causing a computer to perform the functions of:

an obtaining means for obtaining data of a diagnostic image having anatomical structures obtained by imaging a subject, such as a human body or the like, to which a diagnosis is to be given;

a candidate image obtaining means for obtaining a candidate image from the data of the diagnostic image, the candidate image having a candidate region of abnormal shadow included in the diagnostic image;

an anatomical information obtaining means for obtaining anatomical information of the candidate region by detecting an anatomical structure included in the candidate image;

a characteristic amount obtaining means for obtaining characteristic amounts from the candidate region of abnormal shadow included in the candidate image, the characteristic amounts indicating a likelihood of the candidate region being an abnormal shadow;

a retrieving means for retrieving an abnormal shadow image from an image database storing medical images, each having an anatomical structure with an abnormal shadow, obtained by imaging a predetermined region of a subject, such as a human body or the like, the abnormal shadow image being a medical image with an abnormal shadow having comparable anatomical information and characteristic amounts to the anatomical information and characteristic amounts of the candidate region obtained from the candidate image; and

a display means for comparably displaying the candidate region and the abnormal shadow image retrieved from the image database.

The referent of “human body or the like” as used herein may include an animal body, such as a cat or a dog, as well as a human body.

The referent of “structure” as used herein means the tissue forming a predetermined region of a subject.

The referent of “anatomical information” as used herein means information obtainable from each of the tissues for its location, construction, and the like. For example, the chest region is composed of various tissues including ribs, soft-tissue parts, heart, and the like. The information is provided according to the locations of the tissues. That is, it is provided according to, for example, the locations either with or without a rib, or according to the positions within the lung fields (e.g. upper or lower part) that include ribs and soft-tissue parts.

The image display apparatus of the present invention may further comprises an abnormal shadow detecting means for detecting a candidate region of abnormal shadow from the diagnostic image. In this case, the candidate image obtaining means may be configured to obtain the candidate image having the candidate region detected by the abnormal shadow detecting means.

When the medical and diagnostic images are chest images, each obtained by imaging the chest of a human body, the anatomical information obtaining means is preferable to further comprises: a lung field identifying means for identifying lung fields on the chest image, and a rib identifying means for identifying the ribs on the chest image, and the anatomical information obtaining means is configured to obtain anatomical information from the chest image according to the positions within the lung fields and the locations of the ribs.

According to the present invention, an abnormal shadow image having comparable anatomical information and characteristic amounts to those included in the diagnostic image to which a diagnosis is to be given is retrieved from an image database. Then the candidate region and the abnormal shadow image retrieved from the image database are comparably displayed. This facilitates the determination of whether the candidate region is an abnormal shadow or not.

If the apparatus of the present invention is configured to automatically detect a candidate region of abnormal shadow from the diagnostic image, a candidate region which is highly likely an abnormal shadow is detected automatically, as well as that found by the doctor, and determined if it is an abnormal shadow or not by comparing it with an abnormal shadow image. This may reduce the number of overlooked candidate regions of abnormal shadow, and at the same time the determination accuracy may be improved.

When the diagnostic image is a chest image obtained by imaging the chest of a human body, displaying an abnormal shadow according to the positions within the lung fields and the locations of the ribs by identifying the lung fields and ribs allows the images having comparable background images may be displayed, and accurate determination may be made.

### BRIEF DESCRIPTION OF THE DRAWINGS

FIG. 1 is a schematic block diagram of the image display apparatus according to an embodiment of the present invention.

FIG. 2 is a drawing illustrating segmentation of lung fields.

FIG. 3A to 3C are drawings illustrating identification of ribs.

FIG. 4 is a drawing illustrating segmentation of lung fields by ribs.

FIG. 5A is a drawing illustrating a sample chest image having a lung cancer (first example).

FIG. 5B is a drawing illustrating a sample chest image having a lung cancer (first example).

FIG. 6A is a drawing illustrating a sample chest image having a lung cancer (second example).

FIG. 6B is a drawing illustrating a sample chest image having a lung cancer (second example).

FIG. 7 is a flowchart illustrating the operation of the image display apparatus of the present invention.

### DESCRIPTION OF THE PREFERRED EMBODIMENTS

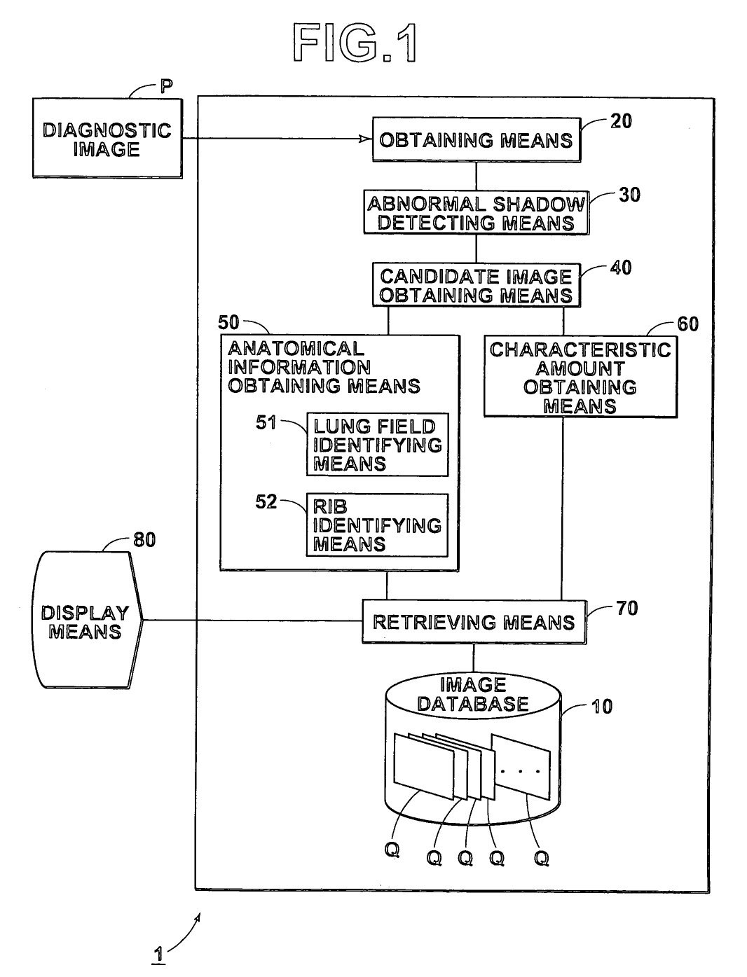

Hereinafter, the image display apparatus according to an embodiment of the present invention will be described with reference to FIG. 1. As shown in FIG. 1, a comparable image display apparatus 1 according to the present embodiment comprises: an image database 10 for storing in advance medical images Q, each having an abnormal shadow, obtained by imaging a predetermined region of a subject, such as a human body or the like; an obtaining means 20 for obtaining data for a diagnostic image P that includes an anatomical structure obtained by imaging a subject, such as a human body or the like, to which a diagnosis is to be given; an abnormal shadow detecting means 30 for detecting a candidate region of abnormal shadow; a candidate image obtaining means 40 for obtaining a candidate image that includes the candidate region of abnormal shadow detected by the abnormal shadow detecting means; an anatomical information obtaining means 50 for obtaining anatomical information by detecting an anatomical structure included in the candidate image; a characteristic amount obtaining means 60 for obtaining characteristic amounts from the candidate region of abnormal shadow included in the candidate image, which indicate a likelihood of the candidate region being an abnormal shadow; a retrieving means 70 for retrieving an abnormal shadow image having comparable anatomical information and characteristic amounts to those obtained from the candidate region; and a display means 80 for comparably displaying the candidate image and the abnormal shadow image retrieved from the image database.

The medical image obtained by imaging a predetermined region of a subject, such as a human body or the like, includes anatomical structures, such as bones, blood vessels, and the like. For example, if the medical image is a chest image obtained by imaging the chest region of a human body, it includes the lung fields with the ribs superimposed thereon, giving anatomically different characteristics depending on the positions within the lung fields or positions with or without a rib. The abnormal shadow like a lung cancer looks differently depending on whether it appears on a rib or a place without a rib. Therefore, in the present embodiment, a specific case example is assumed in which the diagnostic image P and medical image Q are chest images, each obtained by imaging the chest region of a human body, and an abnormal shadow like a lung cancer appeared on the chest image P. Based on this assumption, a method for retrieving comparable images to the abnormal shadow will be described in detail.

The anatomical information obtaining means 50 obtains anatomical information that identifies the position within the lung fields, if the position is on a rib or between the ribs, and the like. Thus, the anatomical information obtaining means 50 has a lung field identifying means 51 for identifying the lung fields and a rib identifying means 52 for identifying the ribs.

Hereinafter, each of the aforementioned means will be described in detail.

(1) Abnormal Shadow Detecting Means

A nodule of lung cancer appearing on the chest image shows different characteristics from those of normal background images.

The abnormal shadow detecting means 30 generates an emphasized image with the nodular being emphasized using an emphasis filter or the like on the diagnostic image P, and a suppressed image with the nodular being suppressed using a suppression filter or the like. Then it obtains a differential image between the emphasized and suppressed images, which is an image with the background image being removed and the nodular being emphasized. The differential image emphasizes not only the nodule but also the structures included in the background images. But more pixels having grey levels (densities) in a predetermined range appear on the nodule, and the nodule shows specific characteristics depending on the size and shape, which are different from those of the structures included in the background images. Consequently, a candidate region of abnormal shadow may be extracted from the background images using characteristic amounts that indicate characteristics appearing on a lung cancer nodule or the like (refer to, for example, “Automated detection of nodules in peripheral lung fields” by M. L. Giger, et al., Med. Phys, 15(2), pp. 158-166, 1988 for detail).

The abnormal shadow, such as a nodule or tumor found on a cancerated part, included in the chest image has basically a rounded contour, and is observed as a region to which many gradient vectors concentrate, since it has greater pixel values than the surrounding area. This type of abnormal shadow is observed as a roundly protruding region, that is, a hemispherical region in which pixels having the same density value are extending concentrically. The roundly protruding region has a gradient of pixel values, in which they are distributed such that the pixel value increases (i.e. density value decreases) gradually from the periphery toward the center of the region. The grade lines concentrate toward the center of the abnormal shadow. Thus, the abnormal shadow may be detected by calculating gradient vectors from the gradients of pixel values and based on the concentration level of the gradient vectors.

Thus, the abnormal shadow on the diagnostic image P may be emphasized using, for example, an Iris filter or appropriate ring filter for emphasizing the round protruding region by evaluating the concentration level of the gradient vectors (refer to, for example, a non-patent literature “Convergence Index Filter for Detection of Lung Nodule Candidates” by Jun Wei, et al., IEICE Journal vol. J83-D-II No. 1, pp 118-125, January 2000 for further detail). In addition, a nodule of lung cancer is characterized that it is very small with a high roundness level and a high brightness value. So, the emphasized region may be determined if it is a candidate region of abnormal shadow based on the size, roundness level, brightness value and the like.

Further, an abnormal shadow tends to have more pixels having density levels that fall within a certain range. Therefore, only a malignant shadow may be detected by the following steps as described for example, in Japanese Unexamined Patent Publication Nos. 9(1997)-167238 and 2002-074325. First, a candidate region of abnormal shadow is detected using an Iris filter or the like. Then, a histogram of densities within the candidate region is obtained based on the detected candidate region of abnormal shadow, and a plurality of characteristic amounts based on the histogram, that is, variance value, contrast, angular moment and the like, are calculated. Thereafter, each of the characteristic amounts is defined by a predetermined weighting function to obtain a new evaluating function value, which is used for determining whether the candidate region is a malignant shadow or not.

The characteristic amount obtaining means 60 obtains quantitative measures such as those used for detecting a candidate region of abnormal shadow described above as characteristic amounts. More specifically, (1) size of the candidate region (effective diameter, area), (2) shape of the candidate region (roundness level), (3) density pattern, (4) statistic amounts of pixel values (average, RMS), (5) statistic amounts of textures (spatial frequency analysis, Fourier transform, wavelet transform, etc.) and the like may be used as the characteristic amounts.

(2) Lung Field Identifying Means

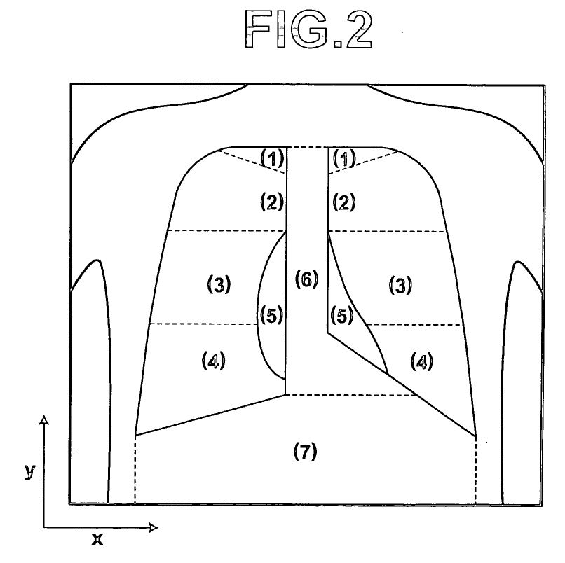

The lung field identifying means 51 identifies the lung fields by detecting the thoracic cage on the chest image P. First, a rough contour of the thoracic cage is extracted using an edge detecting mask, such as the Gabor function or the like, to obtain the approximate center of the thoracic cage. Then, with reference to the center, the thoracic cage is transformed into a polar coordinate system. On the polar coordinate surface, the contour of the thoracic cage is automatically detected by performing a template matching process using a template which is analogous to the reference contour of average thoracic cage contour. The region surrounded by the contour of the thoracic case is identified as the lung fields. Then, as shown in FIG. 2, each of the following seven regions is extracted based on the lung fields (refer to, for example, Japanese Unexamined Patent Publication No. 2003-006661 filed by the applicant of the present invention for further detail). Namely, they are apical lung regions (portions with reference numeral 1), upper lung regions (portions with reference numeral 2), middle lung regions (portions with reference numeral 3), lower lung regions (portions with reference numeral 4), hilar regions (portions with reference numeral 5), mediastinal region (portion with reference numeral 6), and abdominal region (portion with reference numeral 7).

Alternatively, the lung fields are identified and divided into the respective regions using the method described in U.S. Pat. No. 6,549,646.

(3) Rib Identifying Means

The rib identifying means 52 identifies the ribs within the lung fields identified by the lung field identifying means 51. For example, an edge detecting method or Hough transform method (for detecting an ellipse) is used to detect a line or a curve extending substantially straight or gradually curved upwardly in the horizontal direction to a certain extent within the lung fields. Then, each of the detected lines is evaluated to determine the likelihood of the line being a rib. The likelihood is determined based on whether the detected shape corresponds to the location and shape of a rib according to the value that represents the characteristics of the detected convex shape (e.g. location of the ellipse, radius, or the like) as described, for example, in a non-patent literature “Computer Graphics And Image Processing, 7,375-390 (1978).

Alternatively, a statistical model having normal structures without any abnormal shadow may be generated in advance with sample chest images used as teaching data. Thereafter, a rib profile corresponding to that of the inputted chest image P may be generated artificially from the model.

First, images having clearly imaged ribs are selected as the sample images from multitudes of chest images. Then, the points on the ribs on each sample image are used as the teaching data to create the model in advance using a pointing device, such as a mouse or the like. In this way, any rib profile may be generated from the model.

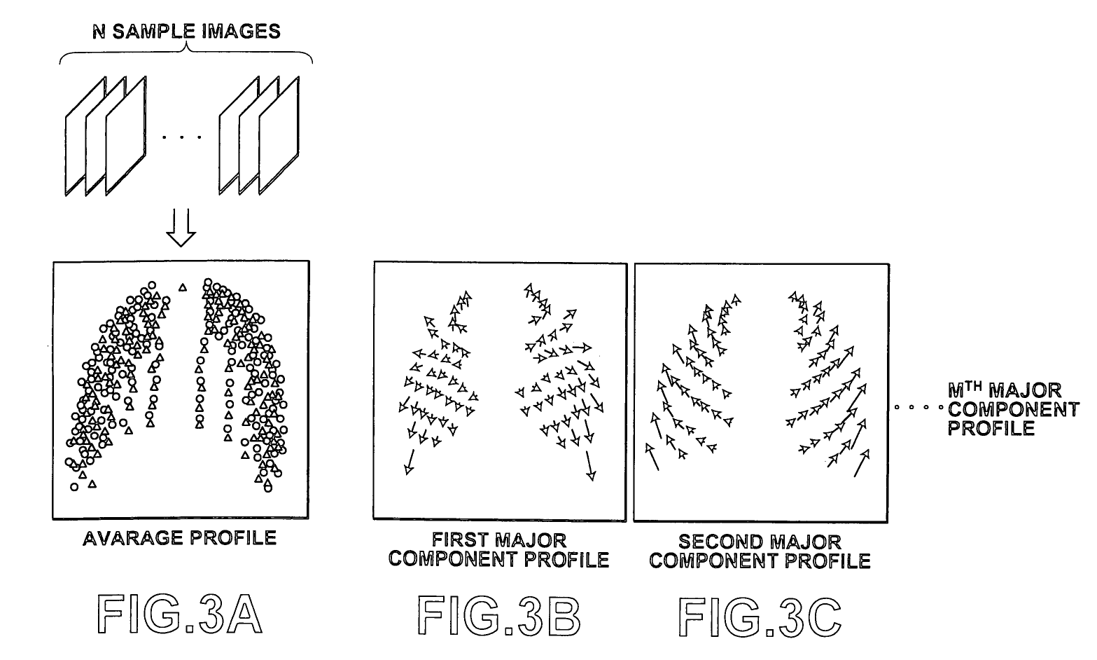

More specifically, an average rib profile Xave of N sample images is obtained (circle marks in FIG. 3A indicate points on the front ribs and triangular marks indicate those on the rear ribs). Then, the differential vector ΔX between the rib profile X of each sample image and the average rib profile Xave (ΔX=X-Xave) is obtained. Then, a major component analysis is performed for differential vector ΔXj to obtain major component vectors for the first to m^{th} major components as shown in FIGS. 3A, 3B and 3C. Thereafter, the rib profile that corresponds to that of the chest image for which ribs are to be detected may be generated by warping the average rib profile Xave using the major component vectors. For example, as shown in FIGS. 3A, 3B and 3C, if the first major component profile P1 appears as a component that extends the profile in the direction indicated by the arrows in FIG. 3B, while the second major component profile P2 appears as a component that extends the profile in the direction indicated by the arrows in FIG. 3C, a rib model having an arbitrary profile may be approximated by the sum of the average profile Xave and each major component profile Ps (s=1, 2 and so on to m) expressed in the following formula.

$$X = Xave + {\sum }_{s}^{m} bsPs$$

By changing the profile factor bs indicated in the formula, the average rib profile may be warped to various different rib profiles. For example, in order to make the average rib profile correspond to that of the chest image P, points on the ribs detected from the chest image P (specifically, points on the rear ribs detected by an edge detection method or the like may be used) maybe substituted to the formula to obtain the profile factor bs and generate a rib profile for identifying the ribs as described, for example, in Japanese Unexamined Patent Publication No. 2004-041694 filed by the applicant of the present invention.

(4) Anatomical Information Obtaining Means

The anatomical information obtaining means 50 divides the lung fields as described herein below based on the lung field and rib identification results described above to obtain the anatomical information.

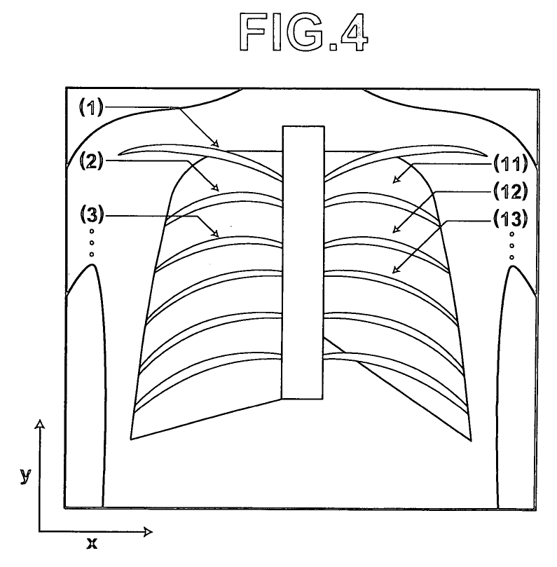

First, it divides the left and right lung fields in the following manner as shown in FIG. 4.
(1) collarbone
(2) first rib
(3) second rib
(4) third rib
(5) fourth rib
(6) fifth rib
(7) sixth rib
(8) seventh rib
(9) eighth rib
(10) ninth rib
(11) between collarbone and first rib
(12) between first and second ribs
(13) between second and third ribs
(14) between third and fourth ribs
(15) between forth and fifth ribs
(16) between fifth and sixth ribs
(17) between sixth and seventh ribs
(18) between seventh and eighth ribs
(19) between eighth and ninth ribs

In this way, the lung fields are divided into 19 regions. By combining the 19 regions divided based on the location of the ribs with the regions divided by the lung field identifying means, the lung fields are subdivided still further.

For example, the lung fields are divided into detailed anatomical regions by allocating the following identification codes to the identified regions.

| | 4^{th} | 3^{rd} | 2^{nd} | 1^{st} | digit |
|---|---|---|---|---|---|
| right upper lung region | 0 | 1 | 3 | 0 | (hex) |
| on second rib: | | | | | |
| left upper lung region | 1 | 1 | 3 | 0 | (hex) |
| on second rib: | | | | | |
| left middle lung region | 1 | 2 | 6 | 0 | (hex) |
| on fifth rib: | | | | | |
| . | | | | | |
| . | | | | | |
| . | | | | | |

In the example shown above, the assumption is made that the fourth digit represents left/right information that indicates which lung field, left or right, has an abnormal shadow, the third digit represents lung field information that indicates which region within the lung field has the abnormal shadow, the second digit represents rib information that indicates on which rib the abnormal shadow locates, and the first digit represents in-between ribs information that indicates in which intermediate region of ribs the abnormal shadow locates, and a hexadecimal (hex) code is allocated to each of the digits in the following manner.

Fourth digit: left/right information, right=0 (hex), left=1 (hex).

Third digit: lung field information, apical region=0 (hex), upper lung region=1 (hex), middle lung region=2 (hex), lower lung region=3 (hex), . . . , hilar region=4 (hex), . . . .

Second digit: rib information, not on a rib=0 (hex), collarbone=1 (hex), first rib=2 (hex), secondrib=3 (hex), . . . , sixth rib=7 (hex), . . . , eighth rib=9 (hex), ninth rig=A (hex).

First digit: in-between ribs information, not between ribs=0 (hex), between collarbone and first rib=1 (hex), between first and second ribs=2 (hex), . . . , between fifth and sixth ribs=6 (hex), . . . , between eighth and ninth ribs=9 (hex).

Based on the results of lung field and rib identifications, one of the identification codes described above is allocated to each of the pixels included in the chest image P, and the identification code of the pixel included in each abnormal shadow region is obtained as the anatomical information.

In the present embodiment, lung regions are divided according to the collarbone, ribs (2) to (10), and between ribs (11) to (19). But, the lung regions may further divided in the X direction. Further, the lung regions may further divided based on whether or not the abnormal shadow is located at the cross section of the ribs, not only on whether or not it is located on a rib.

Further, the lung fields may be divided into anatomical regions by identifying pulmonary blood vessels.

(5) Image Data Base

The image data base 10 stores medical images Q, each having an image of lung cancer, selected from the chest images obtained in the past. The medical images Q stored in the database 10 include images already diagnosed as a case of lung cancer and not include those which are uncertain if they are a lung cancer case.

The lung field and rib identifying processes are performed on the diagnostic image P to divide the lung fields into the anatomical regions, and to classify the region where the lung cancer is located. Then, the image is stored together with the identification code that corresponds to the region where the lung cancer is located.

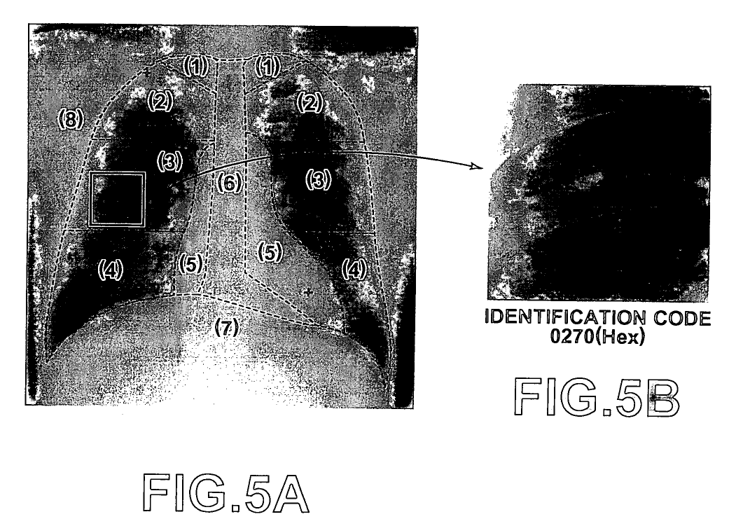

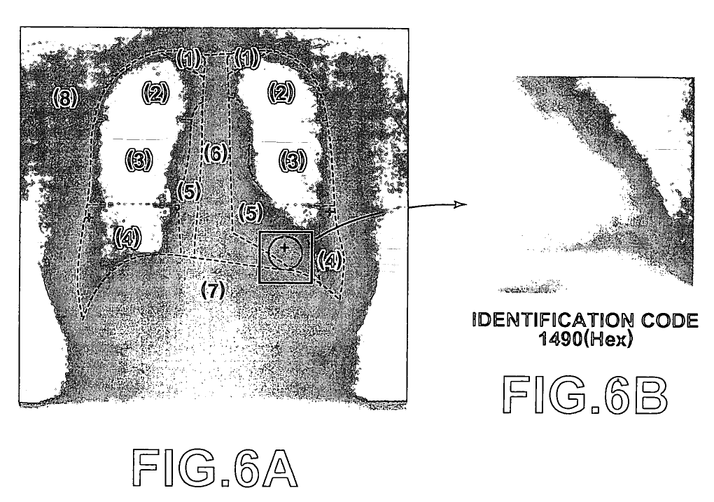

FIGS. 5A and 6A show chest images, each having a shadow of lung cancer of similar malignancy grading (regions enclosed by frames) with each other, and FIGS. 5B and 6B respectively show enlarged view thereof (each reference numeral enclosed by a dotted line corresponds to each region shown in FIG. 2). The lung cancer shown in FIGS. 5A and 5B is developed on the sixth rib in the middle region of right lung field and allocated the identification code of 0270 (hex), while, the lung cancer shown in FIG. 6A and 6B is developed on the eighth rib in the lower region of left lung fields and allocated the identification code of 1490 (hex). When the area where the lung cancer is developed includes a plurality of identification codes, the code that is predominant in the area is used as the identification code. Each lung cancer image is processed by the various processes identical to those described in the detection of abnormal shadow to obtain the characteristic amounts, which are stored with the image.

Preferably, an abnormal shadow image of new case is stored in the image database 10 as it occurs.

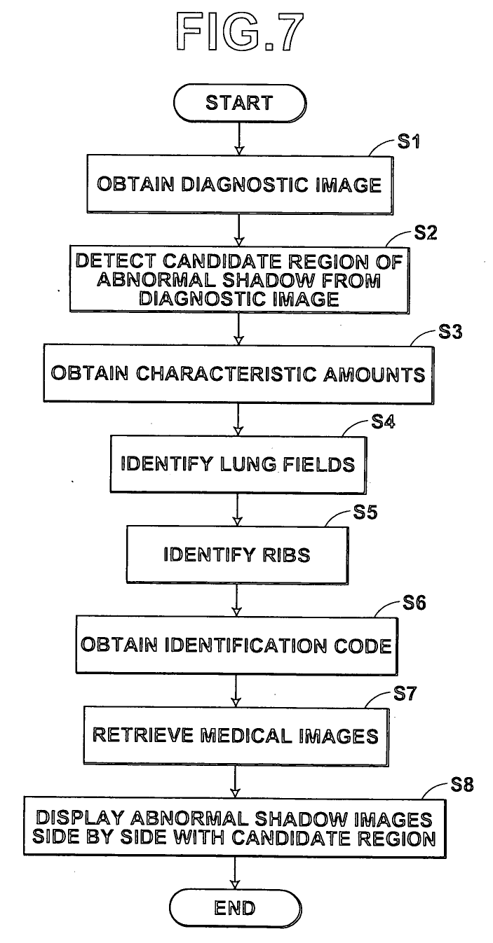

Hereinafter, the operation of the image display apparatus1 of the present embodiment will be described with reference to the flowchart shown in FIG. 7.

First, the image display apparatus1 obtains a diagnostic subject image P from a modality connected to a network through the obtaining means 20. Alternatively, the diagnostic image P may be obtained from a DVD storing the image (step 1). A candidate region of abnormal shadow is detected by the abnormal shadow detecting means 30 from the diagnostic image P obtained in step S1 (step 2). A candidate image that includes the candidate region is received by the characteristic amount obtaining means 60 from the candidate image obtaining means 40, and the size, roundness level, density pattern, statistic amounts of pixel values, statistic amounts of textures and the like for the candidate region calculated by the abnormal shadow detecting means 30 when detecting each candidate region are extracted by the characteristic amount obtaining means 60 as the characteristic amounts (step 3)

Then, the thoracic cage is detected by the lung field identifying means 51 from the chest image P to identify the lung field regions (step 4). Further, ribs within the lung field regions are identified by the rib identifying means 52 (step 5). Based on the results of these, the chest image P is divided into a plurality of anatomical regions by the anatomical information obtaining means 60 to allocate the identification codes thereto, and an identification code that appears most often among the pixels included in the candidate region detected by the abnormal shadow detecting means 30 is obtained as the identification code for the candidate region (step 6).

A medical image Q with an abnormal shadow of case having one of the several similarity determining items (characteristic amounts) that corresponds to the item preset on the retrieving means 70 is retrieved from the image database 10 by the retrieving means 70 (step 7). Preferably, the retrieving means 70 is constructed such that the similarity determining item which has been set thereto may be changed by selecting a desired item from those displayed on the monitor 80.

For example, if the “shape of shadow” is selected, characteristic amounts, such as the “roundness level” and the like which indicate the “shape of shadow”, are inputted to the retrieving means 70 for retrieving the abnormal shadow images having comparable characteristic amounts. The similarity may be determined based on a plurality of similarity determining items, not just on a single item. If the similarity is to be determined based on a plurality of similarity determining items, candidate region may be classified into a plurality of clusters using a discriminator (e.g. SVM (support vector machine), Mahalanobis distance, neural network, or the like) as described, for example, Japanese Unexamined Patent Publication Nos. 9(1997)-167238 and 2002-074325 filed by the applicant of the present invention, and the abnormal image belonging to a particular cluster may be retrieved from the image database.

Then, the abnormal shadow image having the identification code corresponding to that of the candidate region is selected from the images that have been retrieved, and displayed on the monitor of the display means 80 together with the candidate region arranged side by side. When a plurality of abnormal shadow images comparable to the candidate region is retrieved, they are displayed sequentially. Alternatively, the plurality of abnormal shadow images may be displayed simultaneously (step 8).

The lung cancers appeared on the FIG. 5B and 6B have the characteristic amounts indicating comparable malignancy grading, but have different densities depending on whether they are located on a bony part or between the ribs. As described above, shadows appearing on the regions having different anatomical characteristics have different densities so that an accurate determination may not be made by comparing them with abnormal shadow images retrieved based simply on the characteristic amounts. But, an accurate determination may be made by displaying a comparable image having corresponding anatomical characteristics as well as characteristic amounts.

In the present embodiment, description has been made in which the abnormal shadow images having comparable characteristic amounts to those of the chest image P are retrieved first from the image database, then the abnormal shadow images having comparable anatomical information to that of the chest image P are selected from the retrieved images based on the identification code. But, the process steps may be reversed and the abnormal shadow images having comparable anatomical information to that of the chest image P are retrieved first, then the abnormal shadow images having comparable characteristic amounts to those of the chest image P are selected from the retrieved images.

Further, in the present embodiment, description has been made in which an abnormal shadow is automatically detected from the diagnostic image. But, the apparatus of the present invention may be configured such that a region having a seemingly abnormal shadow is specified as a candidate image by the observer, such as a doctor or the like, through the monitor. Then the candidate image is obtained through the candidate image obtaining means 40, and comparable abnormal shadow images to the candidate image are detected. In this case, a process identical to that of the abnormal shadow detecting means is performed on the candidate image specified by the observer to obtain the characteristic amounts of the candidate image.

Further, the image display apparatus of the present invention may be a computer, such as a personal computer or the like, having a program for causing the computer to perform the processing described above installed therein from a CD-ROM or through a network.

As has been described in detail, a candidate region of abnormal shadow may be accurately determined whether it is an abnormal shadow or not by detecting abnormal shadows having comparable characteristic amounts and anatomical information to those of the candidate region, and displaying them side by side with the candidate region on the monitor.

What is claimed is:

1. An image display apparatus, comprising:
an image database for storing medical images, each having an anatomical structure with an abnormal shadow, obtained by imaging a predetermined region of a subject, such as a human body or the like;
an obtaining means for obtaining data of a diagnostic image having anatomical structures obtained by imaging a subject, such as a human body or the like, to which a diagnosis is to be given;
a candidate image obtaining means for obtaining a candidate image from the data of the diagnostic image, the candidate image having a candidate region of abnormal shadow included in the diagnostic image;
an anatomical information obtaining means for obtaining anatomical information of the candidate region by detecting an anatomical structure included in the candidate image;
a characteristic amount obtaining means for obtaining characteristic amounts from the candidate region of abnormal shadow included in the candidate image, the characteristic amounts indicating a likelihood of the candidate region being an abnormal shadow;
a retrieving means for retrieving an abnormal shadow image from the image database, the abnormal shadow image being a medical image with an abnormal shadow having comparable anatomical information and characteristic amounts to the anatomical information and characteristic amounts of the candidate region obtained from the candidate image; and
a display means for comparably displaying the candidate region and the abnormal shadow image retrieved from the image database.

2. The image display apparatus according to claim 1, wherein the apparatus further comprises an abnormal shadow detecting means for detecting a candidate region of abnormal shadow from the diagnostic image, and the candidate image obtaining means is configured to obtain the candidate image having the candidate region detected by the abnormal shadow detecting means.

3. The image display apparatus according to claim 1, wherein:
the medical and diagnostic images are chest images, each obtained by imaging the chest of a human body;
the anatomical information obtaining means comprises:
a lung field identifying means for identifying the lung fields on the chest image; and
a rib identifying means for identifying the ribs on the chest image; and
the anatomical information obtaining means is configured to obtain the anatomical information from the chest image according to the positions within the lung fields and the locations of the ribs.

4. The image display apparatus according to claim 2, wherein:
the medical and diagnostic images are chest images, each obtained by imaging the chest of a human body;
the anatomical information obtaining means comprises:
a lung field identifying means for identifying the lung fields on the chest image; and
a rib identifying means for identifying the ribs on the chest image; and
the anatomical information obtaining means is configured to obtain the anatomical information from the chest image according to the positions within the lung fields and the locations of the ribs.

5. An image display method, comprising:
an obtaining step for obtaining data of a diagnostic image having anatomical structures obtained by imaging a subject, such as a human body or the like, to which a diagnosis is to be given;
a candidate image obtaining step for obtaining a candidate image from the data of the diagnostic image, the candidate image having a candidate region of abnormal shadow included in the diagnostic image;
an anatomical information obtaining step for obtaining anatomical information of the candidate region by detecting an anatomical structure included in the candidate image;
a characteristic amount obtaining step for obtaining characteristic amounts from the candidate region of abnormal shadow included in the candidate image, the characteristic amounts indicating a likelihood of the candidate region being an abnormal shadow;
a retrieving step for retrieving an abnormal shadow image from an image database storing medical images, each having an anatomical structure with an abnormal shadow, obtained by imaging a predetermined region of a subject, such as a human body or the like, the abnormal shadow image being a medical image with an abnormal shadow having comparable anatomical information and characteristic amounts to the anatomical information and characteristic amounts of the candidate region obtained from the candidate image; and
a displaying step for comparably displaying the candidate region and the abnormal shadow image retrieved from the image database.

6. An image display method, comprising:
an obtaining step for obtaining data of a diagnostic image having anatomical structures obtained by imaging a subject, such as a human body or the like, to which a diagnosis is to be given;
a candidate image obtaining step for obtaining a candidate image from the data of the diagnostic image, the candidate image having a candidate region of abnormal shadow included in the diagnostic image;
a characteristic amount obtaining step for obtaining characteristic amounts from the candidate region of abnormal shadow included in the candidate image, the characteristic amounts indicating a likelihood of the candidate region being an abnormal shadow;
an anatomical information obtaining step for obtaining anatomical information of the candidate region by detecting an anatomical structure included in the candidate image;
a retrieving step for retrieving an abnormal shadow image from an image database storing medical images, each having an anatomical structure with an abnormal shadow, obtained by imaging a predetermined region of a subject, such as a human body or the like, the abnormal shadow image being a medical image with an abnormal shadow having comparable anatomical information and characteristic amounts to the anatomical information and characteristic amounts of the candidate region obtained from the candidate image; and
a displaying step for comparably displaying the candidate region and the abnormal shadow image retrieved from the image database.

7. A program for causing a computer to perform the functions of:
an obtaining means for obtaining data of a diagnostic image having anatomical structures obtained by imaging a subject, such as a human body or the like, to which a diagnosis is to be given;
a candidate image obtaining means for obtaining a candidate image from the data of the diagnostic image, the candidate image having a candidate region of abnormal shadow included in the diagnostic image;
an anatomical information obtaining means for obtaining anatomical information of the candidate region by detecting an anatomical structure included in the candidate image;
a characteristic amount obtaining means for obtaining characteristic amounts from the candidate region of abnormal shadow included in the candidate image, the characteristic amounts indicating a likelihood of the candidate region being an abnormal shadow;
a retrieving means for retrieving an abnormal shadow image from an image database storing medical images, each having an anatomical structure with an abnormal shadow, obtained by imaging a predetermined region of a subject, such as a human body or the like, the abnormal shadow image being a medical image with an abnormal shadow having comparable anatomical information and characteristic amounts to the anatomical information and characteristic amounts of the candidate region obtained from the candidate image; and
a display means for comparably displaying the candidate region and the abnormal shadow image retrieved from the image database.
